(19) United States
(12) Patent Application Publication
(10) Pub. No.: US 2025/0139773 A1
(43) Pub. Date: May 1, 2025

(54) INFORMATION PROCESSING APPARATUS, INFORMATION PROCESSING METHOD, AND INFORMATION PROCESSING PROGRAM

(71) Applicant: FUJIFILM CORPORATION, Tokyo (JP)

(72) Inventor: Iori IKEDA, Tokyo (JP)

(21) Appl. No.: 18/922,404

(22) Filed: Oct. 22, 2024

(30) Foreign Application Priority Data
Oct. 31, 2023 (JP) 2023-187214

(51) Int. Cl. G06T 7/00; G06T 7/62; G16H 30/40

(52) U.S. Cl. CPC G06T 7/0012; G06T 7/62; G16H 30/40; G06T 2207/30096

(57) ABSTRACT

An information processing apparatus detects regions of body parts and a lesion from a medical image, derives an evaluation value for each of the regions of the body parts overlapping the lesion, based on a degree of certainty of each partial image constituting the lesion in the medical image, and selects the region of at least one body part detected from the medical image based on the evaluation value.

### CROSS-REFERENCE TO RELATED APPLICATIONS

This application claims priority under 35 USC 119 from Japanese Patent Application No. 2023-187214, filed on Oct. 31, 2023, the disclosure of which is incorporated by reference herein.

### BACKGROUND

### 1. Technical Field

The present disclosure relates to an information processing apparatus, an information processing method, and an information processing program.

### 2. Description of the Related Art

WO20/066670A1 discloses a technique of calculating an evaluation value indicating intensity of a lesion in each of a plurality of images of a biological tissue in an organ.

### SUMMARY

Meanwhile, there are known a technique of detecting a region of a body part, such as an organ or an anatomical region, from a medical image obtained by imaging a subject such as a patient by performing image processing on the medical image, and a technique of detecting a lesion from the medical image. These techniques can support a user such as a doctor in interpreting the medical image by presenting the region including the detected lesion to the user. However, in a case in which all the regions including the lesion are presented, a region with relatively low importance is also presented to the user, which may reduce efficiency of the user in interpreting the medical image. Therefore, it is preferable to be able to appropriately select the region including the lesion in the medical image.

The present disclosure has been made in view of the above circumstances, and an object of the present disclosure is to provide an information processing apparatus, an information processing method, and an information processing program with which a region including a lesion in a medical image can be appropriately selected.

According to a first aspect, there is provided an information processing apparatus comprising: at least one processor, in which the processor detects regions of body parts and a lesion from a medical image, derives an evaluation value for each of the regions overlapping the lesion, based on a degree of certainty of each partial image constituting the lesion in the medical image, and selects at least one region detected from the medical image based on the evaluation value.

A second aspect provides the information processing apparatus according to the first aspect, in which the processor selects the region of which the evaluation value is equal to or greater than a threshold value from among the detected regions.

A third aspect provides the information processing apparatus according to the second aspect, in which the processor calculates the threshold value by multiplying a reference value of the evaluation value by a sensitivity magnification.

A fourth aspect provides the information processing apparatus according to the third aspect, in which the reference value of the evaluation value is a maximum value of the evaluation value derived for each of the regions overlapping the detected lesion.

A fifth aspect provides the information processing apparatus according to the third or fourth aspect, in which the processor performs control of displaying a setting screen for a user to set the sensitivity magnification.

A sixth aspect provides the information processing apparatus according to any one of the first to fifth aspects, in which the evaluation value is a value that depends on the degree of certainty and a size of the lesion overlapping the regions.

A seventh aspect provides the information processing apparatus according to any one of the first to sixth aspects, in which, in a case of deriving the evaluation value, the processor performs weighting such that a degree to which the evaluation value increases is higher as the degree of certainty is higher.

An eighth aspect provides the information processing apparatus according to any one of the first to seventh aspects, in which the processor performs control of displaying a selection result of the region based on the evaluation value.

A ninth aspect provides the information processing apparatus according to any one of the first to eighth aspects, in which the processor performs control of highlighting the region with a higher degree of emphasis as the derived evaluation value is higher.

According to a tenth aspect, there is provided an information processing method executed by a processor provided in an information processing apparatus, the method comprising: detecting regions of body parts and a lesion from a medical image; deriving an evaluation value for each of the regions overlapping the lesion, based on a degree of certainty of each partial image constituting the lesion in the medical image; and selecting at least one region detected from the medical image based on the evaluation value.

According to an eleventh aspect, there is provided an information processing program for causing a processor provided in an information processing apparatus to execute a process comprising: detecting regions of body parts and a lesion from a medical image; deriving an evaluation value for each of the regions overlapping the lesion, based on a degree of certainty of each partial image constituting the lesion in the medical image; and selecting at least one region detected from the medical image based on the evaluation value.

According to the present disclosure, it is possible to appropriately select a region including a lesion in a medical image.

### BRIEF DESCRIPTION OF THE DRAWINGS

Exemplary embodiments of the technology of the disclosure will be described in detail based on the following figures, wherein:

FIG. 1 is a block diagram showing an example of a hardware configuration of an information processing apparatus;

FIG. 2 is a block diagram showing an example of a functional configuration of the information processing apparatus;

FIG. 3 is a diagram for illustrating detection results of a region of a body part and a lesion;

FIG. 4 is a diagram for illustrating a derivation result of an evaluation value;

FIG. 5 is a diagram showing an example of a selection result display screen;

FIG. 6 is a flowchart showing an example of region selection processing; and

FIG. 7 is a diagram showing an example of a sensitivity magnification setting screen.

### DETAILED DESCRIPTION

Hereinafter, an embodiment for carrying out the technique of the present disclosure will be described in detail with reference to the drawings.

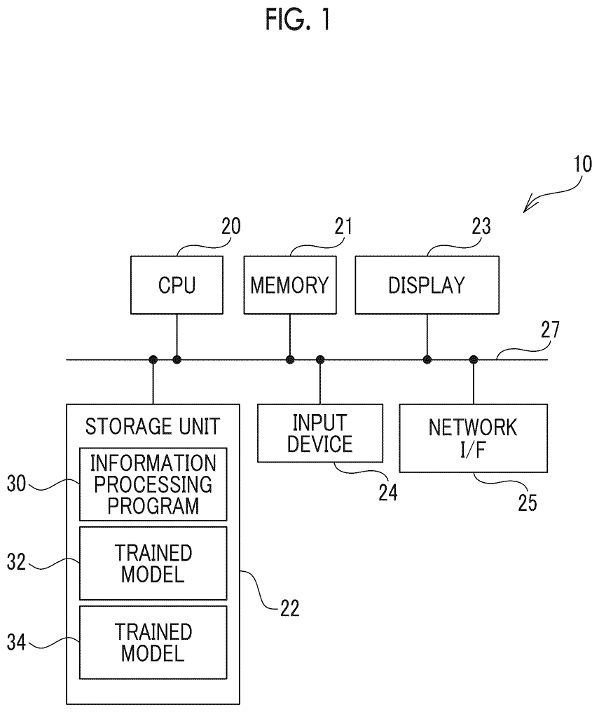

First, a hardware configuration of an information processing apparatus 10 according to the present embodiment will be described with reference to FIG. 1. Examples of the information processing apparatus 10 include a computer such as a personal computer or a server computer. As shown in FIG. 1, the information processing apparatus 10 includes a central processing unit (CPU) 20, a memory 21 as a transitory storage region, and a non-volatile storage unit 22. In addition, the information processing apparatus 10 includes a display 23 such as a liquid crystal display, an input device 24 such as a keyboard and a mouse, and a network interface (I/F) 25 connected to a network. The CPU 20, the memory 21, the storage unit 22, the display 23, the input device 24, and the network I/F 25 are connected to a bus 27. The CPU 20 is an example of a processor according to the technique of the present disclosure.

The storage unit 22 is implemented by a hard disk drive (HDD), a solid state drive (SSD), a flash memory, or the like. An information processing program 30 is stored in the storage unit 22 as a storage medium. The CPU 20 reads out the information processing program 30 from the storage unit 22, loads the readout information processing program 30 in the memory 21, and executes the loaded information processing program 30.

In addition, the storage unit 22 stores a trained model 32 and a trained model 34. The trained model 32 is a model that receives a medical image as input and outputs information representing a region of a body part included in the input medical image. The trained model 32 according to the present embodiment performs labeling of assigning a label corresponding to the body part included in the input medical image to each pixel of a part of a region of the body part, thereby outputting information representing the region of the body part included in the input medical image. The trained model 32 is a model that has been trained through machine learning using a combination of a medical image and information for specifying a region of a body part included in the medical image as learning data.

The trained model 34 is a model that receives a medical image as input and outputs information representing a lesion included in the input medical image. The trained model 34 according to the present embodiment outputs, for each pixel constituting the lesion included in the input medical image, a degree of certainty indicating the likelihood that the pixel is the lesion. The trained model 34 is a model that has been trained through machine learning using a combination of a medical image and information for specifying a region of a lesion included in the medical image as learning data. The trained model 34 may be trained to output a pixel of which the degree of certainty is equal to or greater than a certain value as the region of the lesion.

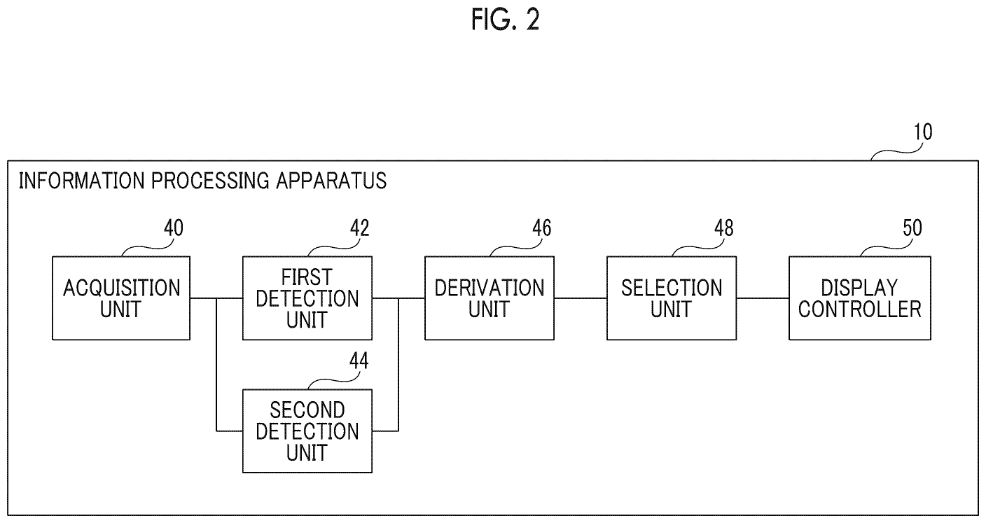

Next, a functional configuration of the information processing apparatus 10 will be described with reference to FIG. 2. As shown in FIG. 2, the information processing apparatus 10 includes an acquisition unit 40, a first detection unit 42, a second detection unit 44, a derivation unit 46, a selection unit 48, and a display controller 50. The CPU 20 executes the information processing program 30 to function as the acquisition unit 40, the first detection unit 42, the second detection unit 44, the derivation unit 46, the selection unit 48, and the display controller 50.

The acquisition unit 40 acquires a medical image of a diagnosis target (hereinafter, referred to as a “diagnosis target image”). For example, the acquisition unit 40 may acquire the diagnosis target image from an external image storage server via the network I/F 25, or may acquire the diagnosis target image from an imaging apparatus that captures a medical image. In addition, for example, in a case in which the diagnosis target image is stored in the storage unit 22, the acquisition unit 40 may acquire the diagnosis target image from the storage unit 22. In the present embodiment, a case in which the diagnosis target image is a chest X-ray image is described as an example.

The first detection unit 42 detects a region of a body part from the diagnosis target image acquired by the acquisition unit 40. Specifically, the first detection unit 42 inputs the diagnosis target image acquired by the acquisition unit 40 to the trained model 32. The trained model 32 outputs information representing a region of a body part included in the input diagnosis target image. As a result, the first detection unit 42 detects the region of the body part from the diagnosis target image. For example, the first detection unit 42 detects, as the region of the body part, a region, such as a left upper lung field, a left middle lung field, a left lower lung field, a left lung hilum part, a right upper lung field, a right middle lung field, a right lower lung field, and a right lung hilum part, from the diagnosis target image. Examples of the region of the body part include an anatomical region and a region of an organ. The first detection unit 42 may detect the region of the body part from the diagnosis target image via a known region detection algorithm.

The second detection unit 44 detects a lesion from the diagnosis target image acquired by the acquisition unit 40. Specifically, the second detection unit 44 inputs the diagnosis target image acquired by the acquisition unit 40 to the trained model 34. The trained model 34 outputs a degree of certainty for each pixel constituting the lesion included in the input medical image. As a result, the second detection unit 44 detects the lesion from the diagnosis target image.

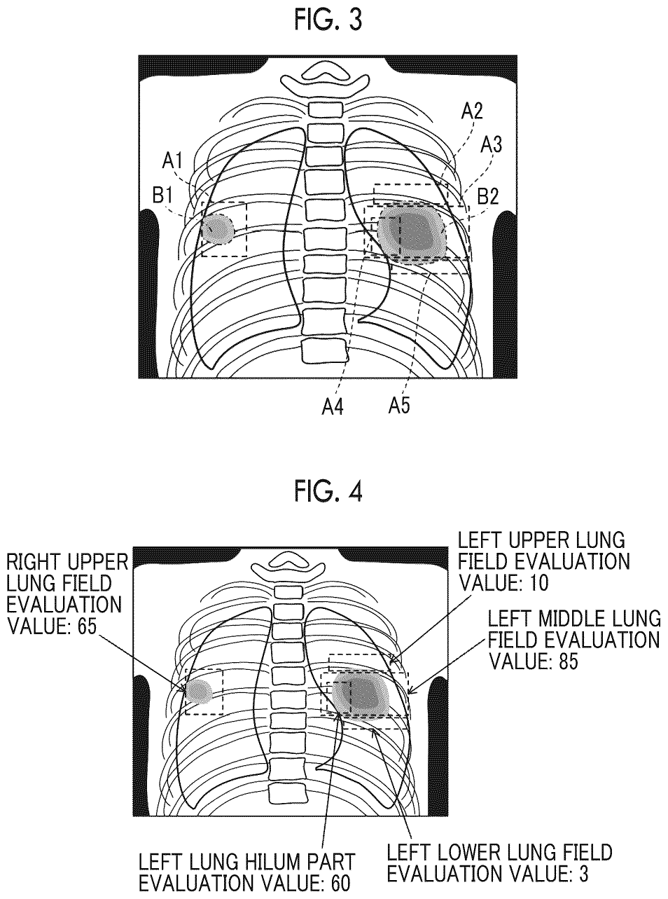

FIG. 3 shows an example of a detection result of the region of the body part via the first detection unit 42 and a detection result of the lesion via the second detection unit 44. In the example of FIG. 3, a portion surrounded by a broken line indicates the region of the body part, and a portion surrounded by a one-dot chain line indicates the lesion. Specifically, in FIG. 3, A1 is a right middle lung field, A2 is a left upper lung field, A3 is a left middle lung field, A4 is a left lung hilum part, and A5 is a left lower lung field. In the example of FIG. 3, a lesion B1 overlaps the right middle lung field A1, and the lesion B2 overlaps the left upper lung field A2, the left middle lung field A3, the left lung hilum part A4, and the left lower lung field A5. In addition, in the example of FIG. 3, the lesion B1 and the lesion B2 are filled with a darker color as the degree of certainty is higher.

The derivation unit 46 derives an evaluation value for each of the regions of the body parts overlapping the lesion based on the degree of certainty of each partial image constituting the lesion detected by the second detection unit 44 in the diagnosis target image acquired by the acquisition unit 40. In the present embodiment, an example in which an image of one pixel is applied as the partial image is described. The partial image may be a plurality of adjacent pixels such as 2×2 pixels.

Specifically, for each lesion in the diagnosis target image, the derivation unit 46 derives an evaluation value for each region by integrating the degree of certainty of each pixel constituting the lesion for each of the regions of the body parts overlapping the lesion. That is, the evaluation value of the region of the body part according to the present embodiment is a value that depends on the degree of certainty of each pixel constituting the lesion and the size of the lesion overlapping the regions of the body parts. In the example of FIG. 3, the integrated value of the degree of certainty of each pixel constituting the lesion B1 is derived as the evaluation value of the right middle lung field A1. In addition, in the example of FIG. 3, the integrated value of the degree of certainty of each pixel constituting the left upper lung field A2 among pixel groups constituting the lesion B2 is derived as the evaluation value of the left upper lung field A2. In addition, in the example of FIG. 3, the integrated value of the degree of certainty of each pixel constituting the left middle lung field A3 among pixel groups constituting the lesion B2 is derived as the evaluation value of the left middle lung field A3. In addition, in the example of FIG. 3, the integrated value of the degree of certainty of each pixel constituting the left lung hilum part A4 among pixel groups constituting the lesion B2 is derived as the evaluation value of the left lung hilum part A4. In addition, in the example of FIG. 3, the integrated value of the degree of certainty of each pixel constituting the left lower lung field A5 among pixel groups constituting the lesion B2 is derived as the evaluation value of the left lower lung field A5.

As an example, as shown in FIG. 4, the evaluation value is derived for each of the regions of the body parts overlapping the lesion for each lesion by the derivation unit 46.

In a case of deriving the evaluation value, the derivation unit 46 may perform weighting such that a degree to which the evaluation value increases is higher as the degree of certainty is higher. In this case, for example, the derivation unit 46 may perform weighting on the degree of certainty by using a quadratic function or an exponential function. Specifically, the derivation unit 46 may use a sigmoid function to perform the weighting such that the evaluation value significantly increases in a case in which the degree of certainty is equal to or greater than a certain value.

The selection unit 48 selects a region of at least one body part detected from the diagnosis target image based on the evaluation value derived by the derivation unit 46. Specifically, the selection unit 48 selects the region of the body part of which the evaluation value derived by the derivation unit 46 is equal to or greater than a threshold value, from among the regions of the body parts detected by the first detection unit 42.

In the present embodiment, the selection unit 48 calculates the threshold value by multiplying a reference value of the evaluation value by a set sensitivity magnification. In addition, in the present embodiment, the selection unit 48 uses the maximum value of the evaluation value derived by the derivation unit 46 for each of the regions of the body parts overlapping the lesion detected by the second detection unit 44, as the reference value of the evaluation value. In addition, the sensitivity magnification is set to a value of 0 or more and 1 or less.

In this way, the selection unit 48 selects the region of the body part of which the evaluation value us equal to or greater than the threshold value, and therefore can appropriately select the region including the lesion in the diagnosis target image, compared to a case in which the regions of all the body parts overlapping the lesion are selected. In addition, since the maximum value of the evaluation value is used as the reference value of the evaluation value and the sensitivity magnification is a value of 0 or more and 1 or less, even in a case in which the lesion overlaps only the region of one body part, the region of the one body part can be selected.

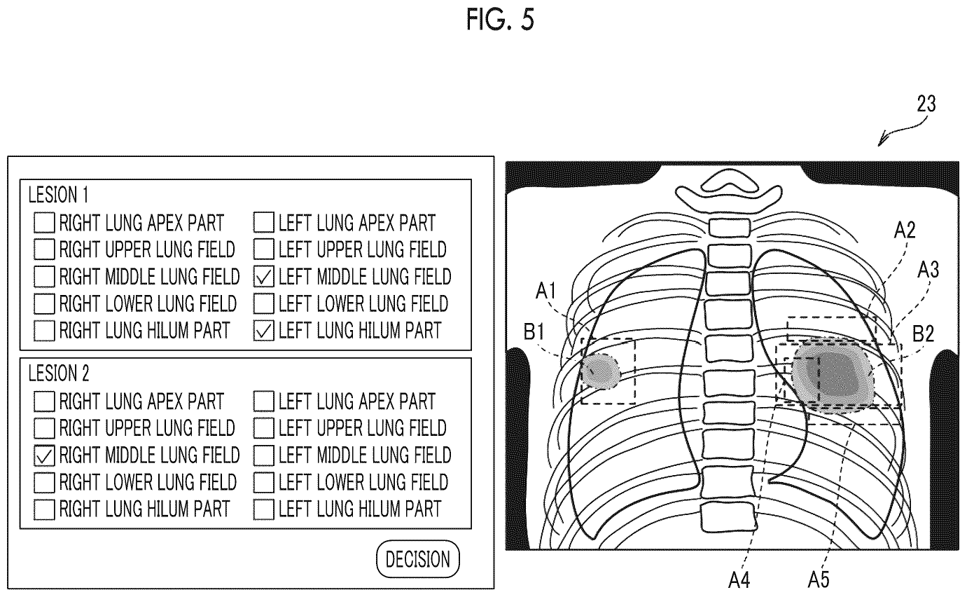

The display controller 50 performs control of displaying a selection result of the region of the body part based on the evaluation value via the selection unit 48 on the display 23. FIG. 5 shows an example of a selection result display screen displayed on the display 23 under the control of the display controller 50. As shown in FIG. 5, the display controller 50 performs control of displaying the selection result on the display 23 together with the diagnosis target image. For example, the display controller 50 performs control of displaying, as the selection result, a name of each of the regions of the body parts detected by the first detection unit 42 and a check box corresponding to each the regions of the body parts for each lesion. In this control, the display controller 50 performs control of displaying the region of the body part selected by the selection unit 48 in a distinguishable manner by assigning a check mark only to the check box corresponding to the region selected by the selection unit 48 among the regions of the body parts detected by the first detection unit 42. In the example of FIG. 5, a lesion 1 corresponds to the lesion B2, and a lesion 2 corresponds to the lesion B1.

As shown in FIG. 5, in the present embodiment, only the left middle lung field A3 and the left lung hilum part A4, which are considered to be relatively important, are selected among all the regions overlapping the lesion B2. A user such as a doctor may interpret the diagnosis target image and assign a new check mark to the check box, or may remove the check mark assigned to the check box.

In a case in which the user performs an operation to decide the selection result of the region of the body part (an operation of pressing a decision button in the example of FIG. 5) on the selection result display screen, for example, a sentence based on the selection result, such as “lesion is present in left middle lung field”, is transcribed into an interpretation report. On the selection result display screen, the display controller 50 may perform control of displaying the degree of certainty of the lesion detected by the second detection unit 44 in the diagnosis target image in a distinguishable manner. For example, the display controller 50 may perform control of highlighting the lesion in a color set as a color that is more conspicuous to the user as the degree of certainty of the lesion in the diagnosis target image is higher. Such a display is also referred to as a heat map display.

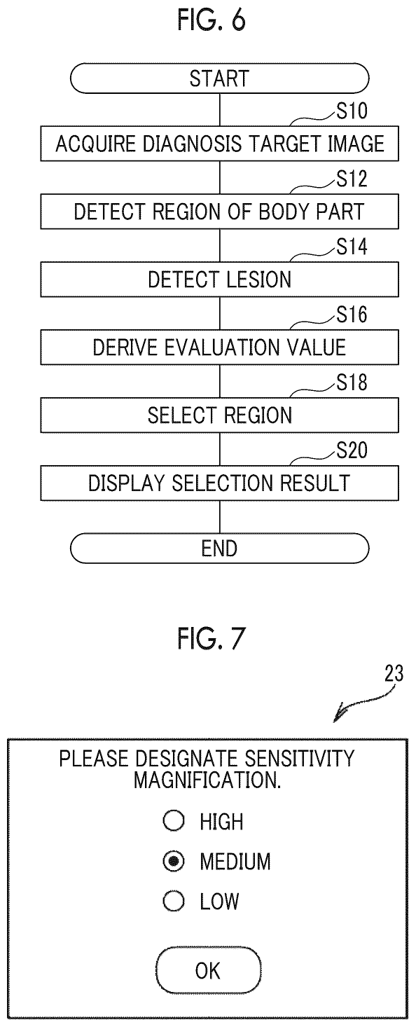

Next, an action of the information processing apparatus 10 will be described with reference to FIG. 6. Region selection processing shown in FIG. 6 is executed by the CPU 20 executing the information processing program 30. The region selection processing shown in FIG. 6 is executed, for example, in a case in which an instruction to start execution is input by the user.

In step S10 of FIG. 6, the acquisition unit 40 acquires the diagnosis target image. In step S12, the first detection unit 42 detects the region of the body part from the diagnosis target image acquired in step S10, as described above. In step S14, the second detection unit 44 detects the lesion from the diagnosis target image acquired in step S10, as described above. The execution order of step S12 and step S14 may be switched, or step S12 and step S14 may be executed in parallel.

In step S16, as described above, the derivation unit 46 derives the evaluation value for each of the regions of the body parts overlapping the lesion based on the degree of certainty of each partial image constituting the lesion detected in step S14 in the diagnosis target image acquired in step S10. In step S18, the selection unit 48 selects the region of at least one body part detected from the diagnosis target image based on the evaluation value derived in step S16, as described above.

In step S20, as described above, the display controller 50 performs control of displaying the selection result of the region of the body part based on the evaluation value in step S18 on the display 23. In a case in which the process of step S20 ends, the region selection processing ends.

As described above, according to the present embodiment, it is possible to appropriately select the region including the lesion in the medical image.

In the above-described embodiment, as an example, as shown in FIG. 7, the display controller 50 may perform control of displaying a setting screen for the user to set the sensitivity magnification on the display 23. In the example of FIG. 7, the sensitivity magnification can be set in three stages of “high”, “medium”, and “low”. For example, in a case in which “high” is designated by the user, the sensitivity magnification is set to 0.7, in a case in which “medium” is designated, the sensitivity magnification is set to 0.5, and in a case in which “low” is designated, the sensitivity magnification is set to 0.3. The number of stages of the sensitivity magnification is not limited to three stages, and may be two stages or five or more stages. In addition, for example, the display controller 50 may perform control of displaying, on the display 23, a setting screen on which the user can input a value equal to or greater than 0 and equal to or less than 1 as the sensitivity magnification. The setting screen in this case may be a screen on which the user can directly input a numerical value corresponding to the sensitivity magnification, or may be a screen on which the user can designate a numerical value corresponding to the sensitivity magnification by moving a slider on a slide bar.

In addition, in the above-described embodiment, the display controller 50 may perform control of highlighting, on the display 23, the region of the body part corresponding to the evaluation value with a higher degree of emphasis as the evaluation value derived by the derivation unit 46 is higher. In this case, for example, the display controller 50 may highlight the name of the region of the body part in the display region of the selection result shown in the example of FIG. 5. In addition, for example, the display controller 50 may highlight the region of the body part in the diagnosis target image shown in the example of FIG. 5. The highlighting includes, for example, a display of making the region of the corresponding body part more conspicuous than other regions by surrounding the region with a bounding box. In addition, the highlighting includes a display of surrounding the region of the body part with a frame line of a color that is preset as a color conspicuous to the user as the evaluation value is higher (for example, a brighter color as the evaluation value is higher). In this way, the region of the body part is more conspicuous to the user as the evaluation value is higher, so that the user can easily grasp the region with a high evaluation value.

In addition, in the above-described embodiment, the selection unit 48 may exclude a region where an index value (for example, an area represented by the number of pixels or the like) representing the size of the lesion overlapping the regions of the body parts is equal to or less than a threshold value, from the selection target.

In addition, in the above-described embodiment, for example, various processors shown below can be used as a hardware structure of a processing unit that executes various kinds of processing, such as each functional unit of the information processing apparatus 10. The various processors include, as described above, in addition to a CPU, which is a general-purpose processor that functions as various processing units by executing software (program), a programmable logic device (PLD) that is a processor of which a circuit configuration may be changed after manufacture, such as a field programmable gate array (FPGA), and a dedicated electrical circuit which is a processor having a circuit configuration specially designed to execute specific processing, such as an application specific integrated circuit (ASIC).

One processing unit may be configured of one of the various processors, or may be configured of a combination of the same or different kinds of two or more processors (for example, a combination of a plurality of FPGAs or a combination of the CPU and the FPGA). In addition, a plurality of processing units may be configured of one processor.

As an example in which a plurality of processing units are configured of one processor, first, as typified by a computer such as a client or a server, there is an aspect in which one processor is configured of a combination of one or more CPUs and software, and this processor functions as a plurality of processing units. Second, as typified by a system on chip (SoC) or the like, there is an aspect in which a processor that implements functions of the entire system including the plurality of processing units via one integrated circuit (IC) chip is used. As described above, various processing units are configured by using one or more of the various processors as a hardware structure.

Further, as the hardware structure of the various processors, more specifically, an electric circuit (circuitry) in which circuit elements such as semiconductor elements are combined may be used.

In addition, in the embodiment described above, an aspect has been described in which the information processing program 30 is stored (installed) in the storage unit 22 in advance, but the present disclosure is not limited to this. The information processing program 30 may be provided in a form of being recorded in a recording medium such as a compact disc read only memory (CD-ROM), a digital versatile disc read only memory (DVD-ROM), and a universal serial bus (USB) memory. Further, the information processing program 30 may be downloaded from an external apparatus via a network.

What is claimed is:

1. An information processing apparatus comprising:
a processor,
wherein the processor
detects regions of body parts and a lesion from a medical image,
derives an evaluation value for each of the regions overlapping the lesion, based on a degree of certainty of each partial image constituting the lesion in the medical image, and
selects at least one region detected from the medical image based on the evaluation value.

2. The information processing apparatus according to claim 1,
wherein the processor selects the region of which the evaluation value is equal to or greater than a threshold value from among the detected regions.

3. The information processing apparatus according to claim 2,
wherein the processor calculates the threshold value by multiplying a reference value of the evaluation value by a sensitivity magnification.

4. The information processing apparatus according to claim 3,
wherein the reference value of the evaluation value is a maximum value of the evaluation value derived for each of the regions overlapping the detected lesion.

5. The information processing apparatus according to claim 3,
wherein the processor performs control of displaying a setting screen for a user to set the sensitivity magnification.

6. The information processing apparatus according to claim 1,
wherein the evaluation value is a value that depends on the degree of certainty and a size of the lesion overlapping the regions.

7. The information processing apparatus according to claim 1,
wherein, in a case of deriving the evaluation value, the processor performs weighting such that a degree to which the evaluation value increases is higher as the degree of certainty is higher.

8. The information processing apparatus according to claim 1,
wherein the processor performs control of displaying a selection result of the region based on the evaluation value.

9. The information processing apparatus according to claim 1,
wherein the processor performs control of highlighting the region with a higher degree of emphasis as the derived evaluation value is higher.

10. An information processing method executed by a processor provided in an information processing apparatus, the method comprising:
detecting regions of body parts and a lesion from a medical image;
deriving an evaluation value for each of the regions overlapping the lesion, based on a degree of certainty of each partial image constituting the lesion in the medical image; and
selecting at least one region detected from the medical image based on the evaluation value.

11. A non-transitory computer-readable storage medium storing an information processing program executable by a processor provided in an information processing apparatus to execute a process comprising:
detecting regions of body parts and a lesion from a medical image;
deriving an evaluation value for each of the regions overlapping the lesion, based on a degree of certainty of each partial image constituting the lesion in the medical image; and
selecting at least one region detected from the medical image based on the evaluation value.
